(19) United States
(12) Patent Application Publication
(10) Pub. No.: US 2023/0373946 A1
(43) Pub. Date: Nov. 23, 2023

(54) PROCESS TO PREPARE A CYCLIC CARBONATE

(71) Applicant: NEW GREEN WORLD B.V., Gouda (NL)

(72) Inventors: Frank VERGUNST, Gouda (NL); Paul VELDER, Gouda (NL); Nicolaas Thomas VAN DER HORST, Gouda (NL)

(73) Assignee: NEW GREEN WORLD B.V., Gouda (NL)

(21) Appl. No.: 18/031,229

(22) PCT Filed: Nov. 10, 2021
(86) PCT No.: PCT/EP2021/081259

§ 371 Date: Apr. 11, 2023

(30) Foreign Application Priority Data
Nov. 12, 2020 (NL) 2026876

(51) Int. Cl. C07D 317/36; B01J 8/22; B01J 8/26; B01J 8/18; B01J 31/22; B01J 35/02; B01J 37/22

(52) U.S. Cl. CPC C07D 317/36; B01J 8/22; B01J 8/26; B01J 8/1809; B01J 31/2217; B01J 35/026; B01J 37/22; B01J 2208/00637; B01J 2208/00017; B01J 2208/00902; B01J 2231/48; B01J 2531/31; B01J 2531/0216

(57) ABSTRACT

The invention is directed to a process to continuously react a gaseous mixture of an epoxide compound and carbon dioxide in the presence of a heterogeneous catalyst at a pressure of between 0.1 and 0.4 MPa in one or more reactors to a liquid cyclic carbonate product and a gaseous effluent stream comprising unreacted epoxide compound and carbon dioxide. Part of the gaseous effluent is purged from the process and another part of the gaseous effluent is fed to an ejector where the gaseous effluent mixes with gaseous mixture of epoxide compound and carbon dioxide having a pressure which is at least more than 0.3 MPa higher than the pressure of the gaseous effluent. The obtained ejector effluent is fed to the one or more reactors.

The invention is directed to a process to continuously react a gaseous mixture of an epoxide compound and carbon dioxide in the presence of a heterogeneous catalyst in one or more reactors to a liquid cyclic carbonate product and a gaseous effluent stream comprising unreacted epoxide compound and carbon dioxide.

Such a process is described in WO2019/125151. This publication describes a process where propylene oxide is reacted with carbon dioxide to propylene carbonate at a pressure of between 0.1 and 0.5 MPa. The reaction is performed in a slurry of liquid propylene carbonate and a supported dimeric aluminium salen complex which complex is activated by benzyl bromide. The supported aluminium salen complex and the benzyl bromide remains in the reactor vessel and liquid propylene carbonate is discharged from the reactor. Unreacted propylene oxide and carbon dioxide as separated from the propylene carbonate product may be recycled to the reactor. A part of this stream may be purged from the process to avoid build-up of non-reacting compounds. The process is performed at relatively low pressures. Nevertheless it will be required to increase the pressure of the gaseous reactants and the described gaseous recycle before feeding these to the reactor. Such an increase may be performed by means of a compressor. A disadvantage of using a compressor is that it introduces complexity to the process.

The object of the present invention is to provide a more simple process which does not have the disadvantages of the prior art process.

This object is achieved by the following process. Process to continuously react a gaseous mixture of an epoxide compound and carbon dioxide in the presence of a heterogeneous catalyst at a pressure of between 0.1 and 0.4 MPa in one or more reactors to a liquid cyclic carbonate product and a gaseous effluent stream comprising unreacted epoxide compound and carbon dioxide and wherein part of the gaseous effluent is purged from the process and another part of the gaseous effluent is fed to an ejector where the gaseous effluent mixes with a gaseous mixture of epoxide compound and carbon dioxide having a pressure which is at least more than 0.3 MPa higher than the pressure of the gaseous effluent to obtain an ejector effluent which ejector effluent is fed to the one or more reactors.

Applicants found that no compressor or at least a smaller compressor is required by the process according to the invention when making use in the ejector of the gaseous mixture of epoxide compound and carbon dioxide having a pressure which is at least more than 0.3 MPa higher than the pressure of the gaseous effluent. This higher pressure mixture may advantageously be obtained by evaporating liquid epoxide at an elevated pressure and by evaporating liquid carbon dioxide having an elevated pressure stored or provided and mixing the evaporated gaseous components. In this way use is made of the high storage pressures of carbon dioxide to arrive at a process which does not require a compressor or does not require a large capacity compressor.

The reactor configuration and how the reactants are supplied and how the reactants and products are processed may be as described in the afore mentioned WO2019/125151. Preferably the one or more reactors are two or more reactors in series comprising a most upstream reactor and a most downstream reactor and optional intermediate reactors. Preferably two reactors in series are used. To the most upstream reactor the ejector effluent is fed. From every reactor a liquid cyclic carbonate product is discharged. An intermediate gaseous effluent comprising unreacted epoxide compound and carbon dioxide is routed from an upstream reactor to the next downstream reactor in the series of reactors. From the most downstream reactor of the series the gaseous effluent stream comprising unreacted epoxide compound and carbon dioxide is discharged. Such a process wherein the reactors are aligned in series is advantageous because it allows one to position a reactor having a more active catalyst as a downstream reactor, preferably as the most downstream reactor. This will enhance the overall conversion to cyclic carbonate and lower the amount of the epoxide compound in the gaseous effluent. This in turn is advantageous because this will result in that less of the valuable epoxide compound is lost via the purge.

The temperature in the reactor may be between 0 and 200° C. and the pressure is between 0.1 and 0.4 MPa (absolute) and wherein temperature is below the boiling temperature of the cyclic carbonate product at the chosen pressure. At the high end of these temperature and pressure ranges complex reactor vessels will be required. Because favourable results with respect to selectivity and yield to the desired carbonate product are achievable at lower temperatures and pressures it is preferred that the temperature in the one or more reactors is between 20 and 150° C., more preferably between 40 and 120° C., and the absolute pressure is between 0.1 and 0.5 MPa, more preferably between 0.1 and 0.3 MPa. The pressure in an upstream reactor is suitably higher than the pressure in a downstream reactor in a series of reactors. This is advantageous because no special measures, such as compressors or blowers, have to be present to create a flow of the intermediate gaseous effluent from an upstream reactor to a downstream reactor.

Most heterogeneous catalysts will deactivate in time. Suitably a reactor comprising a deactivated catalyst is taken off line and subjected to a catalyst regeneration operation. By taking off line is here meant that no reactants like the epoxide compound and carbon dioxide is supplied to the reactor and that no cyclic carbonate is discharged from the reactor. In other words the reactor does not substantially take part in the process to prepare the cyclic carbonate product. Suitably the catalyst of the most upstream reactor is regenerated by taking this reactor off line such the second reactor in the series becomes the most upstream reactor of the series of reactors. A new reactor comprising regenerated catalyst is connected to the series of reactors as the most downstream reactor. Because the most downstream reactor comprises the most active catalyst a high conversion of epoxide compound is achieved.

Taking a reactor off line and online and changing an upstream reactor to become a downstream reactor at the end of a step may be achieved by operating a set of sequence valves and conduits. The time period of one step may be between 1-30 days, preferably between 2-20 days. In such a period of time cyclic carbonate product may be continuously be prepared in the one or more reactors. Regeneration of deactivated catalyst in the off line reactor may be performed in a shorter time period.

The number of reactors in series as described above is preferably two reactors, one upstream reactor directly coupled to one downstream reactor. In addition one reactor may then be regenerated making a total of three reactors for a reactor train. More reactor trains may be operated in parallel.

The gaseous effluent gaseous comprising unreacted epoxide compound and carbon dioxide is obtained in the most downstream reactor of the series of reactors. Part of the gaseous effluent is purged from the process and another part of the gaseous effluent is fed to the ejector. The part that is purged will typically be small, for example less than 5 vol. % of the gaseous effluent. In this purge unreacted epoxide compound and carbon dioxide will be present and some non-reacting compounds, such as nitrogen and other compounds which may be introduced into the process for example as trace impurities of the epoxide compound and/or carbon dioxide feedstock. The purge is necessary to avoid build up of these non-reacting compounds. Because valuable epoxide compound will be lost from the process it is desired to keep the purge as small as possible. It is preferred to increase the pressure of the gaseous effluent before using the gaseous effluent in the ejector. This is especially advantageous when two or more reactors are used in series. In a preferred line up wherein no pressure increasing means for the intermediate gaseous effluents are present the operating pressure in a downstream reactor will be lower than the pressure in its upstream reactor. This pressure loss is suitably compensated by increasing the pressure of the gaseous effluent. Because the required pressure increase is relatively low, preferably less than 0.1 MPa, the means to increase the pressure may be more simple means than the prior art compressor. Preferably this pressure increase is performed by means of a blower. A blower is much less complicated than a compressor. Alternatively a blower may be present between ejector and the one or more reactors.

The catalyst may be present as a fixed bed in a reactor. Preferably the catalyst is present as a slurry of the heterogenous catalyst and the liquid cyclic carbonate product. The reactors may be any reactor in which the reactants and catalyst can intimately contact and wherein the feedstock can be easily supplied to. The reactor as part of a series of reactors is suitably a continuously operated reactor. To such a reactor carbon dioxide and the epoxide compound may be continuously supplied and liquid cyclic carbonate and a gaseous effluent may be continuously discharged. The reactor may be provided with sparger nozzles to add the gaseous feed compounds to the reactor and agitate the preferred catalyst slurry. Agitation may also be achieved by using for example ejectors or mechanical stirring means, like for example impellers. Such reactors may be of the so-called bubble column slurry type reactor and mechanically agitated stirred tank reactor. In a preferred embodiment the reactor is a continuously operated stirred reactor wherein carbon dioxide and epoxide compound are continuously supplied to the reactor. This feedstock is supplied to the most upstream reactor as the ejector effluent and to the other reactor or reactors as the intermediate gaseous effluent. From this continuously operated stirred reactor part of the cyclic carbonate product is continuously withdrawn as part of a liquid stream and a gaseous effluent or intermediate gaseous effluent is continuously withdrawn comprising unreacted carbon dioxide and epoxide. The reactors of a reactor train of two or more reactors in series are preferably of the same size and design. The reactors of optionally parallel operated reactor trains may be different for each train.

When a fixed bed reactor is used the catalyst will remain in the reactor. When a slurry of a heterogeneous catalyst and cyclic carbonate product is used it is preferred to retain the catalyst in the reactor or return the catalyst to the reactor while part of the liquid cyclic carbonate product is discharged from the reactor. Preferably a volume of liquid cyclic carbonate product is discharged from the reactor or reactors in series which corresponds with the production of cyclic carbonate product in the reactor such that the volume of suspension in the reactor remains substantially the same. The liquid cyclic carbonate may be separated from the slurried heterogeneous catalyst by a filter. This filter may be positioned external of the reactor. Preferably the filter is positioned within the reactor. A preferred filter is a cross-flow filter. For the preferred supported dimeric aluminium salen complex as the catalyst is a 10 μm filter, more preferably composed of a so-called Johnson Screens® using Vee-Wire® filter elements, is preferred. The filter may have the shape of a tube placed vertically in the reactor. The filter may be provided with means to create a negative flow over the filter such to remove any solids from the filter opening.

In the process a liquid cyclic carbonate product may be discharged from every reactor of the one or more reactors which are on-line, ie to which reactants are provided. In this discharged liquid cyclic carbonate dissolved epoxide compound may be present. It is preferred to strip out as much of this dissolved epoxide compound by contacting the liquid cyclic carbonate product with the gaseous carbon dioxide obtained by evaporating liquid carbon dioxide. Suitably stripping is performed before mixing the gaseous carbon dioxide with the epoxide compound. In this manner a cleaned product stream of cyclic carbonate is obtained.

The higher pressure mixture may be obtained by evaporating liquid epoxide at an elevated pressure and by evaporating liquid carbon dioxide having an elevated pressure and mixing the evaporated gaseous components. The liquid epoxide compound is preferably increased in pressure by means of a pump when the liquid epoxide compound is stored r provided at a too low pressure. The resulting pressurised liquid epoxide compound is subsequently increased in temperature and partly evaporated by letting down the pressure. Letting down the pressure may be performed in for example a throttle valve. The partly evaporated epoxide compound is separated from the remaining liquid epoxide compound in a gas-liquid separator. The non-evaporated epoxide compound is suitably recycled to the heat exchanger via a pump. The pressure of the gaseous epoxide compound is preferably between 0.5 and 0.8 MPa.

The starting liquid carbon dioxide may be stored or provided via a pipe line. The liquid carbon dioxide suitably has an elevated pressure of between than 1.4 and 4 MPa. The present process advantageously makes use of this elevated pressure. Evaporation may be performed in a vaporiser wherein a substantially gaseous carbon dioxide is obtained. This gas may be heated in a heat exchanger to a temperature of between 80 and 120° C. before it is used in the preferred stripping of the liquid cyclic carbonate product stream as described above. The pressure of the gaseous carbon dioxide is preferably between 0.5 and 0.8 MPa and more preferably substantially the same as the pressure of the gaseous epoxide compound. This allows that the gaseous carbon dioxide and the gaseous epoxide compound may be combined to obtain the gaseous mixture provided to the ejector of epoxide compound and carbon dioxide having a pressure which is at least more than 0.3 MPa higher than the pressure of the gaseous effluent.

The heterogeneous catalyst may be any catalyst suited to catalyse the reaction of carbon dioxide and an epoxide to a cyclic carbonate and which is suitably activated by a halide compound. More especially heterogeneous catalyst comprising an organic compound containing one or more nucleophilic groups such as quaternary nitrogen halides. A preferred heterogeneous catalyst is a supported dimeric aluminium salen complex and the activating compound is a halide compound.

The supported dimeric aluminium salen complex may be any supported complex as disclosed by the earlier referred to EP2257559B1. Preferably the complex is represented by the following formula:

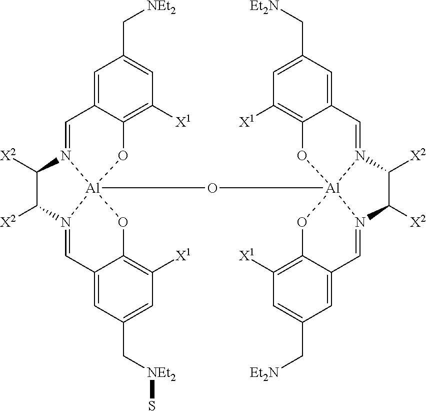

wherein S represents a solid support connected to the nitrogen atom via an alkylene bridging group, wherein the supported dimeric aluminium salen complex is activated by a halide compound. The alkylene bridging group may have between 1 and 5 carbon atoms. X² may be a C6 cyclic alkylene or benzylene. Preferably X² is hydrogen. X¹ is preferably a tertiary butyl. Et in the above formula represents any alkyl group, preferably having from 1 to 10 carbon atoms. Preferably Et is an ethyl group.

S represents a solid support. The catalyst complex may be connected to such a solid support by (a) covalent binding, (b) steric trapping or (c) electrostatic binding. For covalent binding, the solid support S needs to contain or be derivatized to contain reactive functionalities which can serve for covalently linking a compound to the surface thereof. Such materials are well known in the art and include, by way of example, silicon dioxide supports containing reactive Si—OH groups, polyacrylamide supports, polystyrene supports, polyethyleneglycol supports, and the like. A further example is sol-gel materials. Silica can be modified to include a 3-chloropropyloxy group by treatment with (3-chloropropyl)triethoxysilane. Another example is Al pillared clay, which can also be modified to include a 3-chloropropyloxy group by treatment with (3-chloropropyl)triethoxysilane. Solid supports for covalent binding of particular interest in the present invention include siliceous MCM-41 and MCM-48, optionally modified with 3-aminopropyl groups, ITQ-2 and amorphous silica, SBA-15 and hexagonal mesoporous silica. Also of particular interest are sol-gels. Other conventional forms may also be used. For steric trapping, the most suitable class of solid support is zeolites, which may be natural or modified. The pore size must be sufficiently small to trap the catalyst but sufficiently large to allow the passage of reactants and products to and from the catalyst. Suitable zeolites include zeolites X, Y and EMT as well as those which have been partially degraded to provide mesopores, that allow easier transport of reactants and products. For the electrostatic binding of the catalyst to a solid support, typical solid supports may include silica, Indian clay, Al-pillared clay, Al-MCM-41, K10, laponite, bentonite, and zinc-aluminium layered double hydroxide. Of these silica and montmorillonite clay are of particular interest. Preferably the support S is a particle chosen from the group consisting of silica, alumina, titania, siliceous MCM-41 or siliceous MCM-48.

Preferably the heterogenous catalyst is present as a slurry wherein the support S has the shape of a powder having dimensions which are small enough to create a high active catalytic surface per weight of the support and large enough to be easily separated from the cyclic carbonate in or external of the reactor. Preferably the support powder particles have for at least 90 wt % of the total particles a particle size of above 10 μm and below 2000 μm. The particle size is measured by a Malvern® Mastersizer® 2000.

The supported catalyst complex as shown above is activated by a halide compound. The halide compound will comprise a halogen atom which halogen atom may be CI, Br or I and preferably Br. The quaternary nitrogen atom of the complex shown above is paired with the halide counterion. Possible activating compounds are described in EP2257559B1 which exemplifies tetrabutylammonium bromide as a possible activating compound. Benzyl bromide is a preferred activating compound because it can be separated from the preferred cyclic carbonate product, such as propylene carbonate and ethylene carbonate by distillation.

An example of a preferred supported dimeric aluminium salen complex which complex is activated by benzyl bromide is shown below, wherein Et is ethyl and tBu is tert-butyl and Osilica represents a silica support:

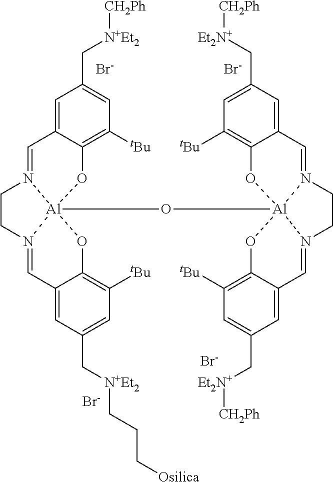

In use the Et group in the above formula may be exchanged with the organic group of the halide compound. For example if benzyl bromide is used as the halide compound to activate the above supported dimeric aluminium salen complex the Et group will be exchanged with the benzyl group when the catalyst is reactivated.

An alternative for the supported dimeric aluminium salen complex as described above may be a supported catalyst wherein an aluminium salen complex part is connected to a support. By positioning these monomers close enough to each other the same catalytic effect as with the dimeric salen complex described above may be achieved. Optionally the supported monomer aluminium salen complex may react with a neighbouring monomer aluminium salen complex to obtain a supported dimeric aluminium salen complex described above which has two connecting bridges to the support instead of one connecting bridge as described above.

The cyclic carbonate product as present in the cleaned product as obtained in the stripper or direct in the reactors may further comprise the activating halide compound. This halide compound is suitably separated from the cyclic carbonate in a distillation step wherein a purified cyclic carbonate product is obtained as a bottom product of the distillation step. The halide activating compound obtained in the distillation step is suitably used to activate a deactivated catalyst, suitably in the off line mode as described above.

It is preferred that the liquid cyclic carbonate product as discharged from the one or more reactors or the cleaned product stream as obtained in the stripper pass a buffer vessel upstream of the distillation step. For a process wherein the heterogeneous catalyst is a supported dimeric aluminium salen complex and the activating compound is a halide compound it is preferred that the volume of the buffer vessel or vessels expressed in m₃ relative to the amount of dimeric aluminium salen complex as present in the one or more reactors, preferably the upstream and downstream reactor, in which the reaction between the epoxide compound and carbon dioxide takes place and expressed in kmol is between 5 and 50 m₃/kmol. Such a buffer vessel will average the content of halide compound in the feed to the distillation column thereby simplifying the distillation operation.

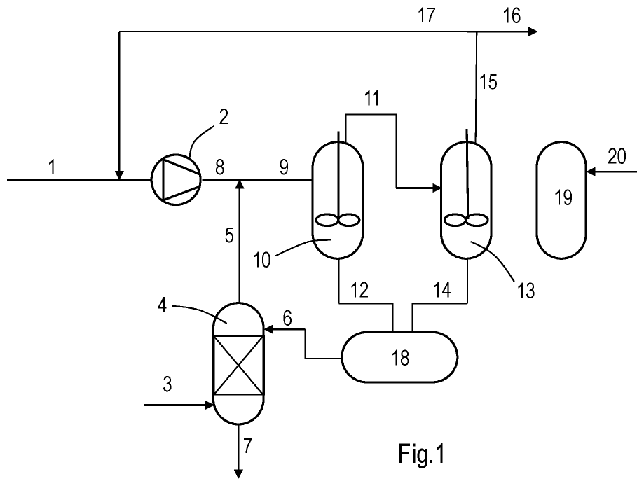

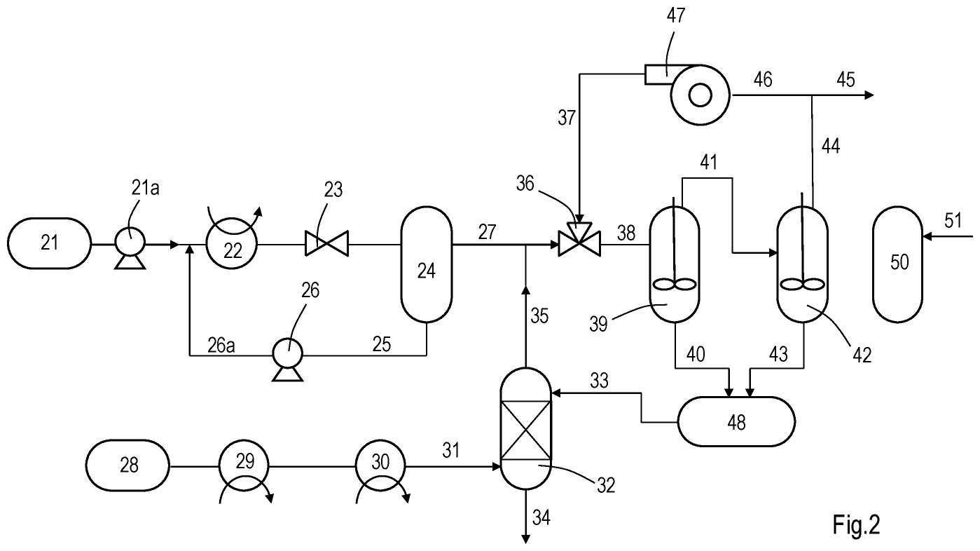

The invention shall be illustrated making use of FIGS. 1 and 2.

FIG. 1 shows a possible line-up for a process not according to the invention to prepare a cyclic carbonate from an epoxide compound and carbon dioxide wherein use is made of a compressor (2) to increase the pressure to the pressure in reactor (10) of a gaseous epoxide compound (1). The epoxide with the increased pressure (8) is mixed with carbon dioxide (5) having about the same pressure. The carbon dioxide (5) contains some epoxide compound which is obtained in stripper (4) by contacting a liquid cyclic carbonate product (6) with gaseous carbon dioxide (3) and wherein a cleaned cyclic carbonate (7) is obtained. The combined epoxide compound and carbon dioxide gaseous mixture (9) is fed to an upstream reactor (10) containing a slurry of a heterogenous catalyst which is activated by a halide compound. From this upstream reactor vessel (10) a first cyclic carbonate product (12) is discharged and an intermediate gaseous effluent (11). The intermediate gaseous effluent (11) is fed to a downstream reactor (13) containing a slurry of the heterogenous catalyst. This reactor (13) is operated at a lower pressure than reactor (10). From this downstream reactor vessel (13) a second cyclic carbonate product (14) is discharged and a gaseous effluent (15). Part of the gaseous effluent (15) is purged as purge (16) and the remaining part of the gaseous effluent (15) is recycled to be combined with the gaseous epoxide compound (1) upstream the compressor (2). The first (12) and second (14) cyclic carbonate streams are collected in a buffer vessel (18). From this vessel a combined liquid cyclic carbonate product (6) is fed to stripper (4). Also shown is a third reactor (19) containing a slurry of the heterogenous catalyst which is regenerated in an off line mode by addition of halide compound (20).

FIG. 2 shows an embodiment according to the invention which does not make use of a large compressor (2) as in FIG. 1. A liquid propylene oxide stored at 16° C. and at 0.2 MPa is increased in pressure by pump (21a) to be mixed with a return flow (26a) of liquid propylene oxide having a temperature of 94° C. and a pressure of 1.3 MPa. The resulting mixture is increased in temperature in heat exchanger (22) to 130 C and reduced in pressure and temperature in throttle valve (23) to a gas (27) and liquid (25) having a pressure of 0.6 MPa and temperature of 95° C. The liquid (25) is recycled via pump (26) to become pressurised return flow (26a).

Liquid carbon dioxide (28) stored at a pressure of 1.9 MPa is regassed in vaporiser (29) and increased in temperature in heat exchanger (30) to a carbon dioxide gas (31) having a temperature of 100° C. and a pressure of 0.6 MPa. In stripper (32) a cleaned propylene carbonate (34) is obtained by contacting a liquid propylene carbonate product (33) with the gaseous carbon dioxide (31). The carbon dioxide (35) as discharged from the stripper (32) contains some reclaimed propylene oxide. This carbon dioxide (35) is combined with the gaseous propylene oxide (27) obtained in the gas liquid separator (24) and the resultant mixture is fed to ejector (36) as the high pressure feed of the ejector having a pressure of 0.6 MPa. To the ejector (36) also a pressurised gaseous effluent (37) having a pressure of 0.23 MPa is fed resulting in an ejector effluent (38) having a pressure of 0.26 MPa. The ejector effluent (38) is fed to the upstream reactor (39) containing a slurry of a heterogenous catalyst which is activated by a halide compound. From this upstream reactor vessel (39) a first propylene carbonate product (40) is discharged and an intermediate gaseous effluent (41). The intermediate gaseous effluent (41) is fed to a downstream reactor (42) containing a slurry of the heterogenous catalyst. This reactor (42) is operated at 0.17 MPa. From this downstream reactor vessel (42) a second propylene carbonate product (43) is discharged and a gaseous effluent (44). Part of the gaseous effluent (44) is purged as purge (45) and the remaining part of the gaseous effluent (46) is increased in pressure in blower (47) to 0.23 MPa to become pressurised gaseous effluent (37). Blower (47) may be considered to be a compressor and will be much smaller than compressor (2) of FIG. 1.

The first (40) and second (43) propylene carbonate streams are collected in a buffer vessel (48). From this vessel a combined liquid propylene carbonate product (33) is fed to stripper (4). Also shown is a third reactor (50) containing a slurry of the heterogenous catalyst which is regenerated in an off line mode by addition of halide compound (51).

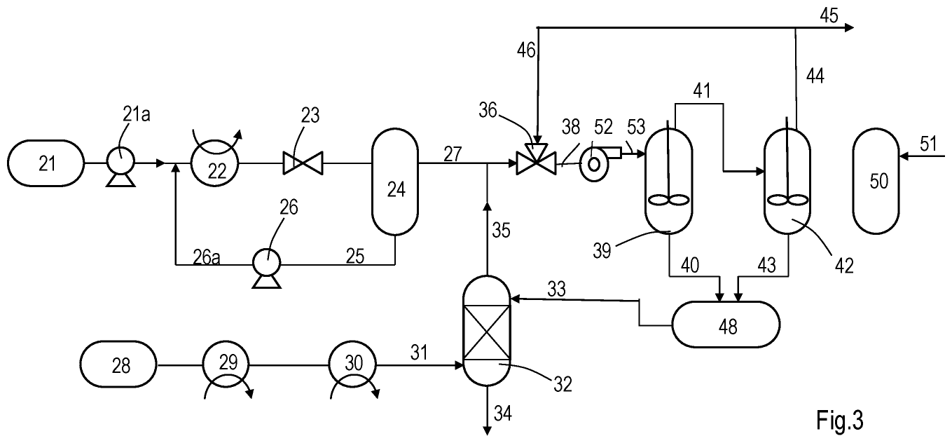

FIG. 3 shows the same embodiment according to the invention as in FIG. 2 except in that the blower is now present downstream of ejector (36). In blower (52) ejector effluent (38) is further increased in pressure before being fed as stream (53) to the upstream reactor (39).

### Comparative Example A

A heat and mass balance is calculated for the process of FIG. 1. The gaseous epoxide is fed at 4.5 kg/s (1 in FIG. 1), the fresh carbon dioxide is fed at 3.5 kg/s (5 in FIG. 1) and the recycle flow is set at 2 kg/s (17 in FIG. 1). The gaseous epoxide and the recycle flow and their resulting mixture upflow compressor (2) has a pressure of 0.7 barg. The energy input for heating feedstock A from 16° C. to 55° C. is calculated. No energy input for CO2 feedstock pressurization (stored at 10+ barg) is taken into account. The required compression duty of compressor (2) of FIG. 1 for compressing the mixture of gaseous epoxide and the recycle from 0.7 barg to the specified reactor inlet pressure 2.1 barg is calculated. For calculation of compression duty, the polytropic compression energy is calculated using a compression efficiency of 65%. The calculated energy consumptions are presented in Table 1.

### Example 1 According to Invention

A heat and mass balance is calculated for the process of FIG. 3. The gaseous epoxide is fed at 4.5 kg/s (27 in FIG. 3), the fresh carbon dioxide is fed at 3.5 kg/s (35 in FIG. 3) and the recycle flow is set at 2 kg/s (46 in FIG. 3). In the energy calculations the energy input for heating the epoxide feedstock from 16° C. to 110° C. (at 100° C., feedstock A vapor pressure is 5 barg) and an additional superheating to 110° C. to prevent unwanted condensation in downstream piping is taken into account. No energy input for CO2 feedstock pressurization (will be supplied and stored at 10+ barg) is taken into account. In static ejector (36 in FIG. 3) stream (46) is pressurized to a resulting discharge pressure. The discharge pressure is calculated based on the pre-defined ratio between flows (27), (35) and (46) and using the figures provided by the supplier of static ejector equipment. The remaining required compression duty for a compressor/blower (52) between elector (36) and upstream reactor (39) is calculated to achieve the same pressure as in the Comparative Example. For calculation of compression duty for (52), the polytropic compression energy is calculated using a compression efficiency of 65%.

Both energy balances are calculated and compared in Table 1.

TABLE 1

| | | Example 1 |
|---|---|---|
| | Comparative | According to |
| Energy consumption | Example A | invention |
| Heating liquid Feedstock A to boiling | 346.8 | 904.8 |
| point [kW] | | |
| Evaporation duty at boiling point [kW] | 2103.6 | 1820.4 |
| Heating gas to discharge temperature [kW] | 29.2 | 68.5 |
| Total heating duty for evaporator unit [kW] | 2479.6 | 2793.7 |
| Possible heat integration [kW] | −230.3 | −662.6 |
| Gas compressor duty [kW] | 491.0 | 197.7 |
| Discharge heater to reactor inlet | 73.1 | 386.0 |
| temperature [kW] | | |
| Total energy duty [kW] | 2813.4 | 2714.8 |
| Net energy gain [kW] | 0 | 98.7 |
| Thermal energy duty [kW] | 2322.5 | 2517.1 |
| Electrical energy duty [kW] | 491.0 | 197.7 |

The presented energy consumptions of Table 1 show that the overall, the energy profit when using the static ejector to boost the recycle flow is equal to 3,7% in this calculation example. Also, the CAPEX costs will be reduced due to the downsizing of the required gas compressor, which is replaced by a relatively cheap static component such as the ejector. And moreover, when applying further heat integration to the total plant, which is overall requiring net cooling duty (exothermic process), the thermal energy duty can be further reduced. In which case the net energy benefit, when using the static ejector, further increases, because the amount of electrical energy duty (which cannot be replaced) is larger in the conventional process.

Applicants found that the process of FIG. 2 consumes less energy. The loss in carbon dioxide caused by operating the stripper at a higher pressure in the process of FIG. 3 has been found to be low and fully compensated by the advantage of not having to use the complex compressor and by the lower energy requirement.

1. A process to continuously react a gaseous mixture of an epoxide compound and carbon dioxide in the presence of a heterogeneous catalyst at a pressure of between 0.1 and 0.4 MPa in one or more reactors to a liquid cyclic carbonate product and a gaseous effluent stream comprising unreacted epoxide compound and carbon dioxide; and wherein part of the gaseous effluent is purged from the process and another part of the gaseous effluent is fed to an ejector where the gaseous effluent mixes with a gaseous mixture of epoxide compound and carbon dioxide having a pressure which is at least more than 0.3 MPa higher than the pressure of the gaseous effluent to obtain an ejector effluent which ejector effluent is fed to the one or more reactors.

2. The process according to claim 1, wherein the gaseous effluent is increased in pressure by means of a blower before mixing the gaseous effluent in the injector.

3. The process according to claim 1, wherein the gaseous mixture of epoxide compound and carbon dioxide as supplied to the ejector is obtained by mixing gaseous epoxide obtained by evaporating liquid epoxide and gaseous carbon dioxide obtained by evaporating liquid carbon dioxide having a pressure of between 1.4 and 4 MPa.

4. The process according to claim 3, wherein a liquid cyclic carbonate product is discharged from the one or more reactors and
wherein any epoxide compound present in the discharged liquid cyclic carbonate product is stripped out by contacting the liquid cyclic carbonate product with the gaseous carbon dioxide wherein a cleaned product stream is obtained.

5. The process according to claim 4, wherein the pressure of the gaseous carbon dioxide is between 0.5 and 0.8 MPa.

6. The process according to claim 1, wherein the one or more reactors are two or more reactors in series comprising a most upstream reactor and a most downstream reactor and optional intermediate reactors,
wherein to the most upstream reactor the ejector effluent is fed,
wherein a liquid cyclic carbonate product is discharged from every reactor and wherein an intermediate gaseous effluent comprising unreacted epoxide compound and carbon dioxide is routed from an upstream reactor to the next downstream reactor in the series of reactors and
wherein from the most downstream reactor of the series the gaseous effluent stream comprising unreacted epoxide compound and carbon dioxide is discharged.

7. The process according to claim 6, wherein the catalyst of the most upstream reactor is regenerated by taking this reactor off line such the second reactor in the series becomes the most upstream reactor of the series of reactors and wherein a new reactor comprising regenerated catalyst is connected to the series of reactors as the most downstream reactor.

8. The process according to claim 6, wherein the heterogenous catalyst is present in the two or more reactors in series as a slurry and
wherein the temperature in the two or more reactors is between 20 and 150° C. and below the boiling temperature of the cyclic carbonate product at the chosen pressure.

9. The process according to claim 1, wherein the heterogeneous catalyst comprises an organic compound containing one or more nucleophilic groups.

10. The process according to claim 9, wherein the nucleophilic group is a quaternary nitrogen halide.

11. The process according to claim 10, wherein the heterogeneous catalyst is a supported dimeric aluminium salen complex and the activating compound is a halide compound.

12. The process according to claim 11, wherein the supported dimeric aluminium salen complex is represented by the following formula: 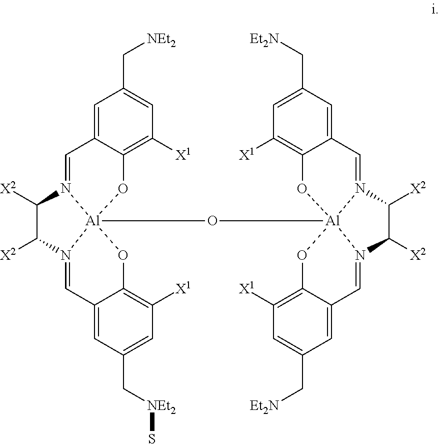
wherein S represents a solid support connected to the nitrogen atom via an alkylene group,
wherein the supported dimeric aluminium salen complex is activated by a halide compound and wherein X¹ is tertiary butyl and X² is hydrogen and wherein Et is an alkyl group having 1 to 10 carbon atoms.

13. The process according to claim 12, wherein the support S is composed of particles having an average diameter of between 10 and 2000 μm.

14. The process according to claim 13, wherein the support S is a particle chosen from the group consisting of silica, alumina, titania, siliceous MCM-41 or siliceous MCM-48.

15. The process according to claim 11, wherein the halide compound is benzyl halide.

16. The process according to claim 15, wherein the benzyl halide is benzyl bromide.

17. The process according to claim 1, wherein the epoxide compound is ethylene oxide, propylene oxide, butylene oxide or pentene oxide.
